(19) United States
(12) Patent Application Publication
(10) Pub. No.: US 2003/0040679 A1
(43) Pub. Date: Feb. 27, 2003

(54) Temperature and body function monitoring mouth guard

(75) Inventors: Christopher Paul Weber, Fort Lauderdale, FL (US); Dane Robert Oleson, Lees Summit, MO (US); Gale Bayard Oleson, Lees Summit, MO (US); Michael Robert Weber, Palm Harbor, FL (US); Paul J. Weber, Fort Lauderdale, FL (US); Luiz B. Da Silva, Danville, CA (US)

Correspondence Address: John P. Wooldridge, 1334 Ridgestone Court, Livermore, CA, 94550, US

(73) Assignee: Pearl Technology Holdings, LLC

(21) Appl. No.: 09/939,979

(22) Filed: Aug. 27, 2001

(51) Int. Cl. A61B 5/00

(52) U.S. Cl. 600/549; 600/301

(57) ABSTRACT

A device is described that can be easily used to warn an athlete of potential risk of hyperthermia due to an elevated core body temperature. The mouth guard device continually monitors the users internal or core body temperature and emits an alarm signal when body temperature exceeds a preset value. This invention can protect the physical well being of an athlete as well as improve overall team performance.

### BACKGROUND OF THE INVENTION

1. Field of the Invention

The present invention relates to protective athletic equipment, and more specifically, it relates to a device and method for monitoring internal or core temperature in an athlete to prevent the risk of hyperthermia.

2. Description of Related Art

Modern athletes at the turn of the second millennium are pushing the limits of their own endurance and their environmental limits even farther in hopes of better performance. It is estimated that two million people play organized football every year at the college, high school, or professional level, as well as, the junior and pee wee levels. Training for these sports can often occur on hot days leading to the possible risk of hyperthermia. Approximately four to six players die each year of hyperthermia.

The most important parameter to monitor on an athlete in order to prevent hyperthermia is the athlete's internal, or core temperature. Measuring the surface skin temperature with stickers, as is done in the anesthesiology setting in an operating room, is impractical and inaccurate. Placing a simple sticker on an athlete's forehead would be impractical and inaccurate because a helmet might cover the area and sweating may occur over the device. Just as in medicine, a true core temperature usually needs to be derived by testing a representative body cavity, such as the mouth, anus and rectum, or the ear canal. These areas are usually exposed to more direct internal body environments. Testing the armpit or nose may reveal inaccurate results, and is usually regarded as an accurate measure of core temperature in the medical community. Therefore, a need exists for a convenient device that can reduce the risk of hyperthermia and be easily used during sports.

There is a need for a device that monitors an athlete's internal or core temperature and can trigger an alarm when that temperature exceeds a preset value. The present invention fulfills this need, and further provides related advantages.

### SUMMARY OF THE INVENTION

An object of the present invention is to provide a device to monitor internal or core body temperature to reduce the risk of hyperthermia in athletes or others.

Another object is to provide a device for wirelessly transmitting information measured by a device that monitors internal or core body temperature to a receiver.

These and other objects will be apparent to those skilled in the art based on the disclosure herein.

The device is a temperature and body monitoring mouth guard worn by an athlete. It both protects the teeth and continuously monitors core body temperature. When the user's core body temperature exceeds a preset value (e.g., 104 F.), an alarm is emitted to warn the athlete or others around him of a potential health hazard. This information can also be wirelessly transmitted to a computer for analysis, making it possible to monitor multiple athletes and improve the overall performance of the team.

An embodiment of the present invention is a mouth guard made of biocompatible material. The users upper and lower teeth fit within the mouth guard and the tongue protrudes in and around the mouth guard. A temperature sensor having minimal or no toxicity is contained in the mouth guard in a location having close proximity to the oral mucosa, the inner side of the mouth or the tongue. This area is relatively representative of core temperature. A flap, bulge or protrusion may be added to bring the surface of the inner mouth guard into apposition separately with the tongue and provide more accurate temperature measurements. A protrusion in the front portion of the mouth guard is an ideal location for a light emitting diode to indicate a health warning. The lips of the user typically do not meet in this area when a large mouth guard is in place making it an easy and visible location to mount a warning mechanism.

The temperature sensor is placed close to the surface to insure quick temperature measurements of the local tissue. The temperature sensor can be placed in a variety of locations as long as it has contact with the tongue or the inner surface of the cheek. Electrical wires connect the temperature sensor to the control electronics. The electronic control circuit is powered by a battery which is turned on and off by the user with a switch to increase battery life. A light can be emitted to indicate that the device is activated. The on/off switch can also be heat activated to turn on when the temperature exceeds a preset value. When the athlete's core body temperature exceeds the preset value, the control circuit activates a warning mechanism to indicate that a dangerous body temperature has been reached. The warning mechanism could be a light emitting diode, a color changing LED, or a bright flashing LED. Alternative alarm indicators can also be used such as an LCD to display current temperature; a vibrating element can be used to signal the user to a potential hazard. The alarm could also emit an audible signal. The LED could also be replaced with a flavor release reservoir, which would be activated when a dangerous temperature is reached. It is important that the temperature response time is quick and accurate allowing the athlete to remove the mouth guard between plays without risking inaccurate measurements.

An RF transmitter can be incorporated into the mouth guard so that the measurements can be communicated to a central receiver, which would allow multiple athletes to be monitored at the same time. It is also possible to replace the temperature sensor with a sensor to monitor blood oxygen, respiratory flow and sound flutter indicative of snoring/sleeping problems. A more advanced embodiment of the present mouth guard utilizes multiple sensors that make the mouth guard useful not only for athletes but also patients while sleeping.

### BRIEF DESCRIPTION OF THE DIAGRAMS

The accompanying drawings, which are incorporated into and form part of this disclosure, illustrate embodiments of the invention and together with the descriptions, serve to explain the principles of the invention.

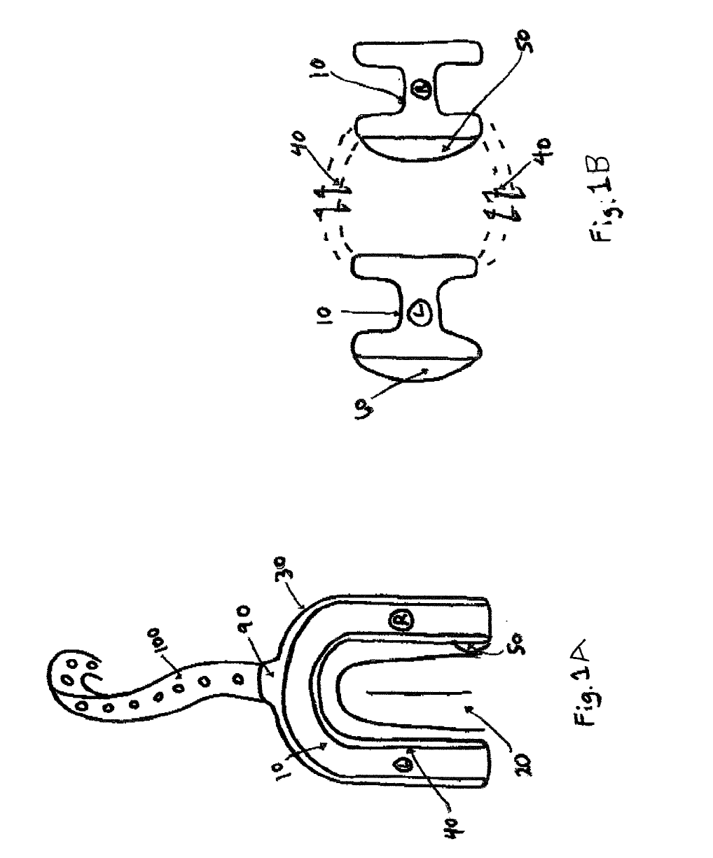

FIG. 1A is a top view of the mouth guard with the tongue in place.

FIG. 1B is a rear view of the mouth guard showing the shapes of the right and left side and the areas where the teeth insert.

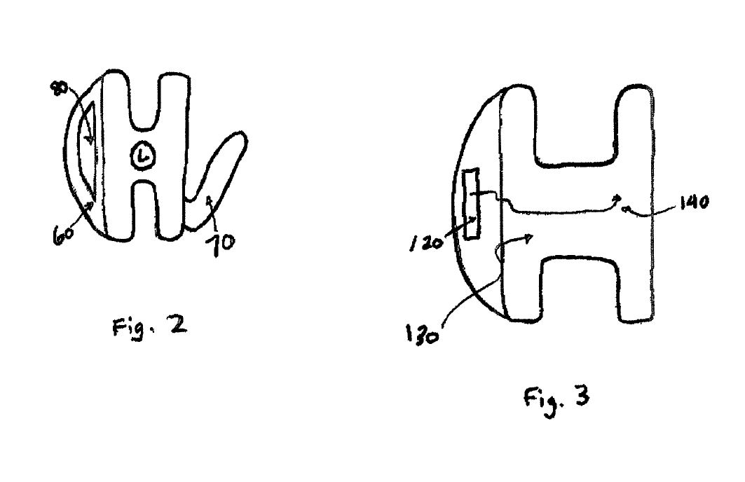

FIG. 2 shows alternate mechanisms for causing protrusion for an element of the mouth guard to come close to contacting either the side wall of the mouth where it is warm and where blood vessel flow is high, or the inner lateral surface of the tongue in which the blood vessel flow is even higher and more representative of a core temperature and core bodily functions.

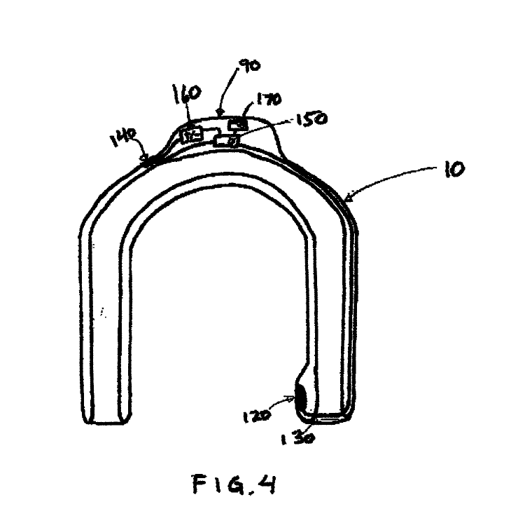

FIG. 3 shows a posterior view of the device and a sample bulge that is made allowing for the sensor to come into closer contact with either the inner surface of the mouth or the tongue, depending on which side is inside or out.

FIG. 4 shows a top view and cutaway view of the mouth guard and the location of the battery microprocessor and stimulus-emitting device.

### DETAILED DESCRIPTION OF THE INVENTION

The invention is a device and method that monitors the internal or core body temperature of an athlete to reduce the risk of hyperthermia. The device is a mouth guard worn by the athlete with at least one integrated sensor. When core body temperature exceeds a preset value, an alarm sounds alerting the athlete and those around him that medical attention is necessary. An alternative embodiment of the mouth guard could contain multiple sensors to measure other body functions and wirelessly transmit this information to a computer for analysis. This information could be used to protect the individual athlete's physical health as well as improve the overall performance of the team.

FIG. 1A shows the key elements of an embodiment of the present mouth guard 10. The human tongue 20 can protrude in and around the mouth guard, which includes an outer peripheral rim 30, which lies exterior to the teeth when the teeth are in the biting position. The inner rim 40 of the mouth guard are near the teeth when the teeth are in the biting position, and it is this inner rim which comes into close proximity to the human tongue. The outer rim 30 comes in close proximity in its rear area to the oral mucosa, which is the inner side of the mouth, which can also be relatively representative of core temperature. A forced bulge or flap 50 is made in the inner lining 40, so that good apposition in measuring of the tongue may occur. The frontal extrusion area or a thickening area 90 of the peripheral rim 30 in which the retaining strap 100 is usually placed, is frequently cut off by athletes. Nonetheless, the protrusion 90 is a relatively comfortable area to mount excess material in the rim 30 because the lips typically do not meet in this area when a large mouth guard is put in place, and no rubbing or irritation occurs. Additionally, this area is usually very visible through the front lips on almost all athletes. This would be the ideal place to put a light-emitting diode, such as a red flashing diode to indicate a warning to another person.

FIG. 1B represents a posterior view of the mouth guard, both the right and left sides, showing the area 10 where the inner depression of the mouth guard is present, where the upper and lower teeth rest in the mouth guard. Inner rim 40 is drawn in a broken fashion to save space. Inner bulge 50 is shown on the posterior aspect of the mouth guard, which helps bring it into close contact with the tongue. Outer bulge 60 is placed in the posterior lateral aspect of the mouth guard in order to bring the mouth guard into close proximity for sensing temperature or other variables of the oral mucosa posteriorly, where they are less affected by breathing or other changes.

FIG. 2 represents an enlarged view of FIG. 1B, showing one segment only, the left posterior segment. This view shows alternative ways to bring the bulge into greater apposition with the oral mucosa or tongue. The bulge 60, which would approximate the oral mucosa in the posterior view, could be enhanced by either a small inflation or insert 80 into a pocket. Additionally, a flap, bulge, protrusion, or any other geometric structure 70 may protrude off, especially the inferior portion of the posterior surface of the inner mouth guard, in order to bring it into apposition separately with the tongue.

FIG. 3 shows a cross sectional view through the posterior portion of the mouth guard showing one possible location for a temperature sensor 120. The temperature sensor 120 is placed close to the surface to insure that the sensor quickly responds to the local tissue temperature that contacts the mouth guard. The temperature sensor could be a thermistor (e.g., KC003T-ND from Keystone Thermometrics Corp, 967 Windfall Road, St Marys Pa.), a thermocouple, or integrated circuit temperature sensor (e.g., LM20, LM34, PC87365 from National semiconductor Corp. 1111 West Bardin Rd, Arlington Tex. 76017) Electrical wires 130 connect the temperature sensor to the control electronics. It is important that the temperature sensor and packaging within the device allow the system to have a temperature response time of less than 10 seconds. This would allow a football player to remove the mouth guard between downs without risking inaccurate measurements.

FIG. 4 shows one embodiment of the present invention. The temperature sensor 120 is located near a wall at the back end of the mouth guard where it ensures good contact with the tongue. In an alternative embodiment the sensor 120 could be placed on the outside surface to contact the inner surface of the cheek. Electrical wires 130 connect the temperature sensor element to the electronic control circuit 150. The electronic control circuit 150 is powered by a battery 160 and turned on by switch 140. The switch 140 is activated by the user before use and deactivated after use. When activated, the switch is recessed to minimize any irritation to the user. In one embodiment when the device is activated the light emitting diode (LED) 170 emits light to alert the user that the device is on and working. In another embodiment the switch 140 is a temperature-activated switch that opens when the temperature exceeds a preset temperature. A switch is desirable to increase battery life. The control circuit 150 includes a comparator to detect when the temperature measured by the sensor exceeds a preset value. When the temperature exceeds the preset value the control circuit 150 activates the light emitting diode 170 to indicate a dangerous temperature has been reached. In one embodiment the color of the LED 170 would change from green to red when the body temperature exceeds the preset value. In order to minimize power and extend battery life the control circuit 150 can flash the LED on and off with a low duty cycle (e.g., 10%).

In one embodiment the LED 170 continues to flash red even if the temperature returns below the preset value. This allows the user to determine if his temperature has been at a dangerous level. Coach's and other team players will be able to see the bright flashing red LED even when the mouth guard is within the mouth. The device can be reset by turning it off and on using switch 140. It is important to note that all the temperature sensor elements shown in FIG. 4 are encapsulated within the biocompatible mouth guard material. This protects the elements and user from direct contact In order to reduce risk, it is important that the materials used in the sensor elements have minimal or no toxicity. Although FIG. 4 shows that most of the device elements are located within the front protrusion 90, the device elements can be placed any where within the mouth guard.

In an alternative embodiment, the LED 170 is replaced by alternative alarm indicators. These could include an LCD or organic light emitting diode display that can be used to display the current temperature. The alarm indicator could be a vibrating element that vibrates to signal the user of potential risk. The alarm could also be an audible alarm that sounds when a hazardous condition exists. Yet another alternative is to replace the LED with a flavor release reservoir in which sweet tastes may be stored or aromatics, such as cinnamon and aldehydes, or irritants such as capsaicin. These may be excreted into the saliva in order to alert the athlete that a hazard exists.

In another embodiment, an RF transmitter is incorporated into the mouth guard and the sensor measurements are communicated to a central receiver that can be used to monitor multiple players simultaneously. Another alternative is to replace the temperature sensor with a different sensor element. For example, a saturated blood oxygen sensor similar to those used in pulsed oximetery could be used to monitor blood oxygen and heart rate. A more advanced mouth guard could incorporate multiple sensors (e.g., temperature, blood oxygen, and heart rate) and be used not only for sports but also for monitoring patients while sleeping. The wireless communication module could replace LED 170 and use simple RF communication or technology now available such as the Blue Tooth chip set.

The above descriptions and illustrations are only by way of example and are not to be taken as limiting the invention in any manner. One skilled in the art can substitute known equivalents for the structures and means described. The full scope and definition of the invention, therefore, is set forth in the following claims.

### Temperature and Body Functiom Monitoring Mouth Guard

Christopher P. Weber (USA)

1 Seneca Road Ft. Lauderdale, Fla. 33308

Dane R. Oleson (USA) 25610 Shady Brook Lane Lees Summit, Mo. 64086

Gale B. Oleson (USA) 25610 Shady Brook Lane Lees Summit, Mo. 64086

Michael Robert Weber (USA) 3573 Fairway Forest Drive Palm Harbor, Fla. 34685

Paul J. Weber (USA) 1 Seneca Road Ft. Lauderdale, Fla. 33308

Luiz B. Da Silva (USA) 1995 Camino Ramon Place Danville, Calif. 94526

We claim:

1. A body function monitor, comprising:
a mouth guard; and
at least one sensor integrated into said mouth guard.

2. The monitor of claim 1, wherein said at least one sensor comprises a temperature sensor.

3. The monitor of claim 1, further comprising an alarm operatively connected to said at least one sensor.

4. The device of claim 1, further comprising an activation switch operatively connected to said sensor.

5. The device of claim 1, further comprising a battery operatively connected to said sensor.

6. The device of claim 1, wherein said at least one sensor comprises a plurality of sensors

7. The device of claim 1, further comprising means for wirelessly transmitting sensor information to a computer for analysis.

8. The monitor of claim 1, wherein said mouth guard includes an inner rim which lies interior to the teeth when the teeth are in the biting position, wherein said inner rim comprises material that extends away from said inner rim to contact a user's tongue, wherein said at least one sensor is positioned within said material that extends away from said inner rim.

9. The monitor of claim 1, wherein said mouth guard includes an outer peripheral rim which lies exterior to the teeth when the teeth are in the biting position, wherein said outer peripheral rim comprises material that extends away from said outer peripheral rim to contact a user's inner cheek, wherein said at least one sensor is positioned within said material that extends away from said outer peripheral rim.

10. The monitor of claim 9, wherein said outer peripheral rim includes a frontal extrusion area that comprises a warning indicator operatively connected to said at least one sensor.

11. The monitor of claim 10, wherein said warning indicator comprises a light-emitting diode.

12. The monitor of claim 1, wherein said at least one sensor is selected from the group consisting of a thermistor, a thermocouple, an integrated circuit temperature sensor, a saturated blood oxygen sensor, a pulse sensor and a vibration sensor.

13. The monitor of claim 1, further comprising control electronics located within said mouth guard and operatively connected to said at least one sensor.

14. The monitor of claim 13, wherein said control electronics include a comparator to detect when temperature measured by said at least one sensor exceeds a preset value.

15. The monitor of claim 1, wherein said activation switch is a temperature-activated switch that opens when temperature exceeds a preset value.

16. The monitor of claim 1, wherein said mouth guard comprises biocompatible material.

17. The monitor of claim 1, wherein said mouth guard further comprises a display operatively connected to said at least one sensor.

18. The monitor of claim 17, wherein said display is selected from the group consisting of an LCD and an organic light emitting diode.

19. The monitor of claim 3, wherein said alarm comprises an indicator selected from the group consisting of a vibrating element that vibrates to signal the user of a potential risk, an audible alarm that sounds when a hazardous condition exists and a flavor release reservoir that excretes a flavor into the saliva of a user in order to alert that a hazard exists.

20. The monitor of claim 1, wherein said mouth guard further comprises an RF transmitter operatively connected to transfer a measurement from said at least one sensor to an RF receiver.
